(19) United States
(12) Patent Application Publication
(10) Pub. No.: US 2024/0268432 A1
(43) Pub. Date: Aug. 15, 2024

(54) ENCAPSULATED BACTERIAL, METHODS AND USES THEREOF

(71) Applicants: Universidade Católica Portuguesa - UCP, Porto (PT); CESPU - Cooperativa De Ensino Superior Politécnico E Universitário, CRL, Gandra Prd (PT)

(72) Inventors: Ana Maria PEREIRA GOMES, Vila Nova De Gaia (PT); Daniela Marlene DA SILVA MACHADO, Santa Eulália De Vizela (PT); Diana Isabel PINTO DE ALMEIDA, Senhora Da Hora (PT); Joana Cristina PACHECO BARBOSA, Paço De Sousa (PT); José Carlos MÁRCIA ANDRADE, Porto (PT)

(73) Assignees: Universidade Católica Portuguesa - UCP, Porto (PT); CESPU - Cooperativa De Ensino Superior Politécnico E Universitário, CRL, Gandra Prd (PT)

(21) Appl. No.: 18/291,260

(22) PCT Filed: Jan. 25, 2022
(86) PCT No.: PCT/IB2022/056846

§ 371 Date: Jan. 23, 2024

(30) Foreign Application Priority Data
Jul. 23, 2021 (PT) 117361

(51) Int. Cl. A23L 33/135; A23G 1/42; A23P 10/30; A61K 9/50; A61K 35/00; A61K 35/741; C12N 1/04

(52) U.S. Cl. CPC A23L 33/135; A23G 1/423; A23P 10/30; A61K 9/5063; A61K 35/741; C12N 1/04; A61K 2035/115

(57) ABSTRACT

The present disclosure related to encapsulated anaerobic probiotics, methods of encapsulation, food compositions and foodstuff.

The present invention is useful in the food industry and allows the maintenance of viable anaerobic bacteria in food products and/or food supplements even if stored in aerobic conditions without refrigeration.

### TECHNICAL FIELD

The present disclosure relates to encapsulated anaerobic probiotics, methods of encapsulation, food compositions and foodstuff.

The present invention is useful in the food industry and allows the maintenance of viable anaerobic bacterium in food products and/or food supplements even if stored in aerobic conditions without refrigeration.

### BACKGROUND

Modern medicine focus on maintaining and improving health through the prevention of diseases rather than treatment of diseases. Gut microbiota plays a key role in modulating an individual's health and can also be used as a biomarker of an individual's general well-being. The human microbiota is an important barrier- and immune-modulator and actively participates in the regulation of the metabolism, intervening in the digestion and nutrient uptake and synthesis processes. In addition, the balance between gut bacterial species, in terms of number and proportion, also contributes to exclude pathogenic bacteria thus decreasing the risk of bacterial infections and the need for antibiotics. Variations in this balance can be found among humans and can be tightly correlated with several diseases. Probiotic bacteria are a key player in the gut health modulation ecosystem. Several bacterial strains have been widely used for human consumption due to their well-established beneficial effects. These known “classical” probiotics include several strains from the genera Bacillus, Lactobacillus, and Bifidobacterium and are usually associated with fermented foods, mainly dairy products. However, other food matrices are currently under consideration, given the percentage of the population that is intolerant to lactose or milk proteins.

Besides advances in the food matrices field, new probiotic candidates are also being studied, with the human gut itself being regarded as a major source of next-generation probiotic strains, including Akkermansia muciniphila, Anaerobutyricum hallii and Faecalibacterium prausnitzii. A major advantage of using such source is that, as commensal bacteria, gut strains are in general considered intrinsically safe for human consumption. Several studies have been performed to characterize these next-generation probiotics and the nature of their interaction with other commensal bacteria and the host. For instance, A. muciniphila has shown promising results in ameliorating metabolic conditions such as type 2 diabetes and obesity by regulating the glucose metabolism and improving gut barrier (Almeida et al 2020). The presence of A. muciniphila in the gut mucus layer induces the proliferation of other mucus-colonizing bacteria due to the production of oligosaccharides and acetate resulting from mucus degradation; thus, the proliferation of pathogenic bacteria is impaired due to competitive exclusion within this layer (Belzer and De Vos 2012).

A problem present in the state of the art is the challenge of working with gut-derived bacteria since they are usually highly sensitive to oxygen and nutritionally demanding. They often demand specific requirements in terms of nutrients, oxygen and pH, which makes them hard to handle under laboratorial and industrial conditions (Clark, 2019). This is also a major challenge when designing delivery methods to make such probiotic strains available to consumers. An adequate method for delivering the probiotic must ensure the administration of adequate amounts of live bacteria and be metabolically active in the consumer's gut. A preferable method for delivering the probiotic can be in the form of a food or beverage matrix that enables easy intake (Šipailienė and Petraitytė, 2018). This method must also guarantee the survival of the probiotic strain during the manufacture of the food product itself and throughout the expected storage period until consumption. It must be suitable for mild storage conditions adapted for shelf-life in the market and at the consumers' household environment. This means it must be suitable for storage across a wide range of temperatures and exposure to atmospheric oxygen.

Several existing solutions to this problem involve the application of encapsulation methodologies to overcome the oxygen susceptibility of gut commensal bacteria, specifically for A. muciniphila.

Van der Ark et al (2017) efficiently encapsulated A. muciniphila MUCT (=DSM 22959, CIP 107961, ATCC BAA-835, JCM 33894) strain in a water-in-oil-in-water double emulsion. Furthermore, these researchers demonstrated that the survival of bacteria encapsulated in the double emulsion was higher than that of free dispersed cells after in vitro gastric and intestinal passage. Nevertheless, encapsulated A. muciniphila suffered a dramatic reduction in viability after only 72 hours of storage at 4° C., either under aerobic or anaerobic conditions.

Marcial-Coba et al (2018) encapsulated A. muciniphila DSM 22959 in a xanthan/gellan gum matrix, via the extrusion method, with a subsequent freeze-drying step, in which various combinations of cryoprotective agents were employed. These researchers verified that the cryoprotective solutions with a high sugar or protein content provided higher bacterial survival during freeze-drying. In addition, the survival rate of freeze-dried microencapsulated A. muciniphila was higher than that of free cells during in vitro simulated gastrointestinal tract passage. However, a significant decrease in freeze-dried microencapsulated A. muciniphila viability was observed upon storage in both anaerobic and aerobic conditions, after 15 and 30 days, at both 4° C. and 25° C., but more evidently for 25° C.

The same research group explored using dark chocolate as a carrier for freeze-dried microencapsulated A. muciniphila in a xanthan/gellan gum matrix in terms of the bacterial's survival during storage and during in vitro simulated gastrointestinal tract transit (Marcial-Coba et al., 2019). Embedding in dark chocolate conferred an increased protection to the encapsulated A. muciniphila since only a slight (although significant) reduction in viability was observed after 60 days of anaerobic storage at 4° C. and 15° C. It also resulted in a high survival rate after in vitro gastric transit at pH 3. It is important to mention that, in a hedonic sensory test, dark chocolate containing microcapsules was not significantly different from two commercially available chocolates.

Chang and colleagues (2020) demonstrated the possibility of using spray-drying for encapsulation of A. muciniphila. This technique is industrially attractive since it involves a quick encapsulation, low cost, good reproducibility and applicable in large-scale such as in the food industry. However, spray-drying involves high temperatures and causes a high osmotic pressure and exposure to oxygen which could be deleterious for the microbial cells. Prior to spray-drying, the authors used succinate-grafted alginate doped with epigallocatechin-3-gallate (EGCG) microgels to encapsulate A. muciniphila strain 139 (obtained from mice). This encapsulation matrix protected A. muciniphila cells during spray-drying and the resulting formulation presented an improved storage in aqueous environments under anaerobic conditions at 4° C. for 12 days and showed an increased survival under simulated gastro-intestinal tract conditions in comparison with spray-dried free cells. However, a decreasing tendency in viability (measured via MTT assay) was observed over the 12 days of storage.

Recently, a commercial product containing next-generation probiotics, such as A. muciniphila, Clostridium beijerinckii, C. butyricum, Bifidobacterium infantis and Anaerobutyricum hallii, and a prebiotic compound (inulin) was employed by Perraudeau and colleagues (2020) and is already in the market to help manage type 2 diabetes (Pendulum Glucose Control—https://pendulumlife.com). This product was tested in a clinical trial and was shown to be safe and well tolerated and improved postprandial glucose control (Perraudeau et al., 2020). No details on the manufacture or stabilization procedures of the product were given since this product is protected by several patents in the USA (U.S. Pat. Nos. 9,173,910; 9,486,487; 10,149,867; 10,149,870; 10,668,116; 10,675,312; among other patents pending).

Existing solutions do not provide efficient methods to protect A. muciniphila during storage at aerobic and non-refrigerated conditions. Further, only the spray-drying procedure seems to be capable of being scaled up to industrial levels. However, the encapsulation matrix and its production are highly complex to perform at industrial level.

These facts are disclosed in order to illustrate the technical problem addressed by the present disclosure.

### General Description

The present disclosure related to encapsulated probiotics, methods of encapsulation, food compositions and foodstuff.

The method of the present disclosure surprisingly allows the maintenance of viable anaerobic bacterium even if stored in aerobic conditions without refrigeration.

An aspect of the present disclosure relates to a scalable method for encapsulation by using protecting agents that are cheaper and easier to manipulate.

Another aspect of the present disclosure relates to the use of several suitable food matrices to ensure the survival of A. muciniphila and putatively other next-generation probiotic candidates, during long term-storage under aerobic conditions and across a wide range of temperatures.

In an embodiment, the final formulation ensures the efficient delivery of an adequate number of viable bacterial cells upon simulated gastrointestinal tract passage.

In an embodiment, Akkermansia muciniphila DSM 22959 was encapsulated using spray-drying technique and with skim milk as a food matrix.

Akkermansia muciniphila DSM 22959 can be purchased in https://www.dsmz.de/collection/catalogue/details/culture/DSM-22959 the bacterium is widely known and used.

In an embodiment, the present disclosure relates to an encapsulated anaerobic bacteria obtained by spray-drying and encapsulation in a solution of milk derived products; preferably milk-derived dry products.

In an embodiment, the encapsulated anaerobic bacteria are selected from Akkermansia muciniphila, Anaerobutyricum hallii, Faecalibacterium prausnitzii or mixtures thereof. (In relation to Faecalibacterium prausnitzii, the strains that were part of this species have been reclassified into 3 new species, so these 3 new species are those that will be—in theory—covered by the previous nomenclature: Faecalibacterium duncaniae sp. nov. (type strain JCM 31915T=DSM 17677T=A2-165T), Faecalibacterium hattorii sp. nov. (type strain JCM 39210T=DSM 107841T=APC922/41-1T) and Faecalibacterium gallinarum sp. nov. (type strain JCM 17207T=DSM 23680T=ic1379T), https://www.microbiologyresearch.org/content/journal/ijsem/10.1099/ijsem.0.005379).

In an embodiment, the encapsulated bacterium is Akkermansia muciniphila DSM 22959.

In an embodiment, the encapsulated anaerobic bacterium is encapsulated in a solution of milk derived products, wherein the solution of milk derived products is a skim milk.

Another aspect of the present disclosure relates to a particle comprising a capsule, wherein said capsule comprises a solution of milk derived products, preferably milk-derived dry products, in the shell and an anaerobic bacterium in the core.

In an embodiment, the capsule is obtainable by spray-drying.

In an embodiment, the bacteria in the particle are selected from: Akkermansia muciniphila; Anaerobutyricum hallii; Faecalibacterium prausnitzii; wherein Faecalibacterium prausnitzii is one of the following: Faecalibacterium duncaniae, Faecalibacterium hattorii, Faecalibacterium gallinarum, or mixtures thereof. (In relation to Faecalibacterium prausnitzii, the strains that were part of this species have been reclassified into 3 new species, so these 3 new species are those that will be—in theory—covered by the previous nomenclature: Faecalibacterium duncaniae sp. nov. (type strain JCM 31915T=DSM 17677T=A2-165T), Faecalibacterium hattorii sp. nov. (type strain JCM 39210T=DSM 107841T=APC922/41-1T) and Faecalibacterium gallinarum sp. nov. (type strain JCM 17207T=DSM 23680T=ic1379T), https://www.microbiologyresearch.org/content/journal/ijsem/10.1099/ijsem.0.005379).

In an embodiment, the bacteria in the particle are Akkermansia muciniphila DSM 22959.

In an embodiment, the quantity of anaerobic bacteria ranges from 10⁵ to 10¹⁰ CFU/g.

In an embodiment, the amount of the solution of milk derived products ranges from 5-20% (v/v).

Another aspect of the present disclosure relates to a foodstuff composition comprising the particle and/or the encapsulated anaerobic bacteria described in the present disclosure and a suitable food matrix and/or viable anaerobic bacteria in a chocolate matrix.

In an embodiment, the suitable food matrix is selected from: dairy products, cereal matrix, oil matrix, sugar matrix, chocolate matrix, soy milk matrix, juice matrix, vegetable matrix and fruit matrix.

In an embodiment, the quantity of particle and/or the encapsulated anaerobic bacteria in the food composition ranges from 10⁶-10¹¹ CFU/serving, preferably 10⁷-10⁹ CFU/serving.

In an embodiment, the bacteria are viable at least after 12 days of aerobic storage conditions at 4° C.-25° C.; preferably wherein the bacteria are viable at least after 4 weeks of aerobic storage conditions at 4° C.-25° C.

In an embodiment, the present disclosure relates to foodstuff comprising the food composition described in the present disclosure.

In an embodiment, the foodstuff comprising the particle and/or the encapsulated anaerobic bacteria of the present disclosure is cheese, yogurt, ice-cream, plant-based yoghurt-type product, beverage, juice, chocolate food supplement, cereal bar.

Another aspect of the present disclosure relates to a method of obtaining the encapsulated bacteria of the present disclosure comprising the steps of:
obtaining a suitable anaerobic bacterium;
adding a solution of milk-derived dry product to the sample of bacterium to obtain a bacteria suspension; preferably the solution of milk-derived dry product is skim milk;
spray-drying the suspension to obtain encapsulated bacteria.

In an embodiment, the bacteria that are used in the method of the present disclosure are selected from Akkermansia muciniphila, Anaerobutyricum hallii, Faecalibacterium prausnitzii or mixtures thereof. (In relation to Faecalibacterium prausnitzii, the strains that were part of this species have been reclassified into 3 new species, so these 3 new species are those that will be—in theory—covered by the previous nomenclature: Faecalibacterium duncaniae sp. nov. (type strain JCM 31915T=DSM 17677T=A2-165T), Faecalibacterium hattorii sp. nov. (type strain JCM 39210T=DSM 107841T=APC922/41-1T) and Faecalibacterium gallinarum sp. nov. (type strain JCM 17207T=DSM 23680T=ic1379T), https://www.microbiologyresearch.org/content/journal/ijsem/10.1099/ijsem.0.005379).

In an embodiment, the bacterium that is used in the method of the present disclosure is Akkermansia muciniphila DSM 22959.

In an embodiment, the spray-drying conditions can be: an inlet temperature from 140-180° C.; an outlet temperature from 55-80° C.; and a flow rate from 5-20 ml/min.

In an embodiment, the milk-derived product that is uses in the method of the present disclosure is a skim milk.

In an embodiment, the encapsulated bacterium is for use as a live biotherapeutic product or food additive.

In an embodiment, the encapsulated bacterium of the present disclosure is for use as a probiotic.

### BRIEF DESCRIPTION OF THE DRAWINGS

The following figures provide preferred embodiments for illustrating the description and should not be seen as limiting the scope of invention.

FIG. 1 shows Akkermansia muciniphila DSM 22959 cell viability, in CFU/g, during Aerobic storage at 4° C. (A) and 23° C. (B) after spray-drying at different operating conditions: inlet (140-180° C.); outlet (55-80° C.); flow rate (5-20 mL/min).

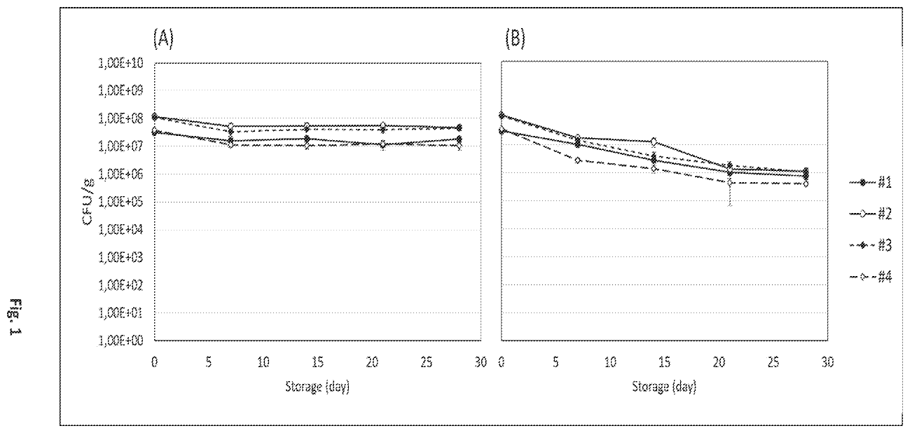

FIG. 2A shows the viability of Akkermansia muciniphila DSM 22959 (spray-dried and free cells) upon incorporation into four food matrices (A). FIGS. 2(B) and 2(C) represent normalized data of A. muciniphila viability during aerobic storage throughout 14 days for “Foodstuff A” and “Foodstuff B” matrices, respectively.

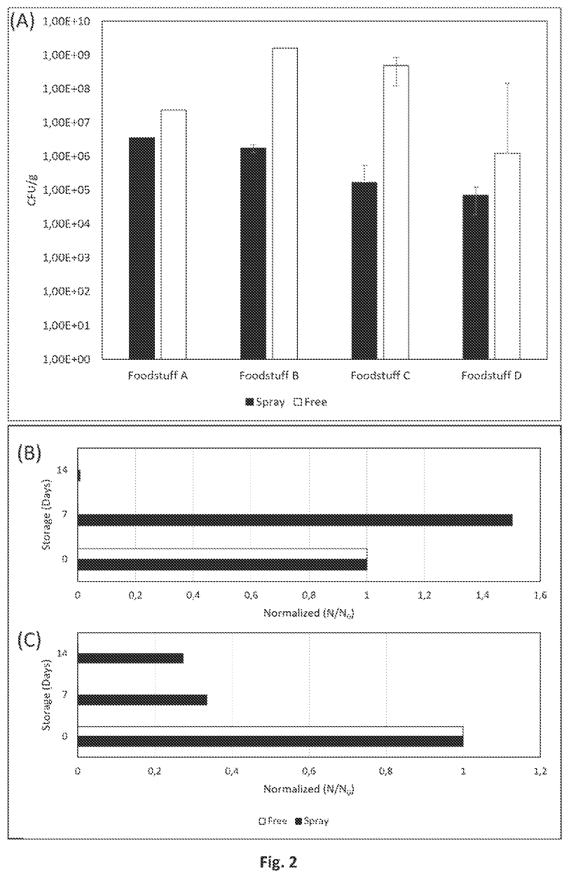

### DETAILED DESCRIPTION

The present disclosure related to encapsulated probiotics and methods of encapsulation.

In embodiment, Akkermansia muciniphila DSM 22959 was microencapsulated using spray-drying technique and with skim milk as a food matrix.

Akkermansia muciniphila DSM 22959 strain was obtained from Leibniz Institute DSMZ-German Collection of Microorganisms and Cell Cultures (Braunschweig, Germany). For long-term storage, this bacterial strain was kept frozen at −80° C. in PYG broth supplemented with 0.05% (m/v) mucin (PYGM, media composition in accordance with DSMZ recommendations (DSMZ, 2021) and with 20% (v/v) glycerol (Fisher Chemical, Loughborough, United Kingdom). A glycerol stock of A. muciniphila DSM 22959 was thawed and grown in PYGM broth for 24 h at 37° C. and under anaerobic conditions (85% N₂, 5% H₂ and 10% CO₂) achieved using an anaerobic incubator (Whitley A35 HEPA anaerobic workstation, Bingley, United Kingdom). After incubation, the bacterial suspension was sub-cultured three times, preferably twice, in PYGM broth before its use to ensure an adequate quantity of viable cells. The resulting cell suspension was centrifuged at 12 000×g, 30 min, 4° C. and washed once with half the volume of sterile PBS. The bacterial pellet was then resuspended into ⅕ of the initial volume of 0.9% (w/v) NaCl. Cell viability was determined by plating 10 μl decimal dilutions onto PYGM agar (1.5%) plates and the suspension was kept at 4° C. until spray-drying.

In an embodiment, Akkermansia muciniphila DSM 22959 bacterial suspension was mixed with a 5 to 20% (w/v), preferably 10% (w/v), solution of milk-derived dry products (preferably skim milk solution, previously sterilized at 121° C. for 5 min) at a final concentration of 5 to 20% (v/v), preferably 10% (v/v). This mixture was dried in a B-290 Mini Spray-Dryer (Buchi, Switzerland) with the conditions set as follows: an inlet temperature of 140 to 180° C., preferably 150° C., an outlet temperature of 55 to 80° C., preferably 65° C., and a flowrate of approximately 5 to 20 mL/min, preferably 7.5 mL/min. The dried powder was then collected, weighed and stored in sterile plastic cups that were sealed and stored at 4° C. until further use. A sample was serial diluted and plated to count the number of CFU that were effectively encapsulated.

The initial suspension, containing free and untreated A. muciniphila cells was used as control in the subsequent incorporation steps.

In an embodiment, the spray-dried and encapsulated Akkermansia muciniphila DSM 22959 bacteria was incorporated into food matrices.

In an embodiment, the spray-dried encapsulated Akkermansia muciniphila DSM 22959 and Akkermansia muciniphila DSM 22959 free cells were incorporated into several food matrices with diverse basic compositions: chocolate-based product, spreadable dairy cream, cereal mixture, and honey. The spray-dried encapsulated Akkermansia muciniphila DSM 22959 cells were incorporated at a final concentration of 5 to 15% (w/w), preferably 10% (w/w); in the case of the incorporation of free A. muciniphila cells, the pellet resulting from the centrifugation of a suspension volume that equals the mass of the matrix was used.

In an embodiment, spray-dried encapsulated as well as free Akkermansia muciniphila DSM 22959 cells were incorporated into a chocolate matrix (50 to 95% (w/w); cocoa), preferably 70% (w/w). The chocolate matrix was prepared as follows: melt the appropriate amount of chocolate for each condition (free or spray-dried) in a water-bath; allow it to cool down to 37° C. and add the respective bacterial preparation; temper at 34° C. for 10 min. Distribute into aliquots of approximately 2 g; allow the aliquots to cool down at 10-11° C., for 2 h; store under aerobic atmosphere at 20° C.

In an embodiment, spray-dried encapsulated as well as free Akkermansia muciniphila DSM 22959 cells were incorporated into a spreadable dairy cream comprising 55 to 85% (w/w), preferably 70% (w/w), pasteurized whey cheese with 5 to 35%, preferably 20%, Greek-type yoghurt; thermal treatment in a water bath at 90° C. for 10 min; allow the mixture to cool down until 37° C. and add the bacterial preparation, mixing thoroughly; split into two containers for each condition (free and spray-dried); store under aerobic atmosphere at 4° C.

In an embodiment, spray-dried encapsulated as well as free Akkermansia muciniphila DSM 22959 cells were incorporated into a cereal mixture bar (comprising fine and coarse bran, wheat germ, xantan gum and water); spray-dried A. muciniphila (or bacterial suspension, in the case of free cells) were added; distributed into aliquots of approximately 2 g each; store under aerobic atmosphere at 20° C.

In an embodiment, spray-dried encapsulated as well as free Akkermansia muciniphila DSM 22959 cells were incorporated into honey by warming up the honey to 37° C. and adding the bacterial preparation (free or spray-dried); split the mixture into two containers for each condition; allow it to cool down and store under aerobic atmosphere at 20° C.

In an embodiment, the spray-dried encapsulated and the free Akkermansia muciniphila DSM 22959 cells were kept in aliquots at 4° C. and 20° C. for viability control over time.

In an embodiment, the viability of A. muciniphila DSM 22959 incorporated into the different food matrices were determined over time in the following timepoints: 0, 7, 14 and 21 days. For food matrices, aliquots were weighed, dissolved in pre-warmed (37° C.) PBS (1:9) and homogenized by vortexing; for spray-dried A. muciniphila controls, 1 g of powder was rehydrated in sterile PBS, to a 1:9 proportion and homogenized by vortexing. Finally, for A. muciniphila free cells, the bacterial suspension was directly diluted and plated. Serial decimal dilutions were performed in sterile PBS and 10 μl of each dilution were spotted, in triplicates, on PYGM agar plates. Plates were incubated at 37° C. for 7 days under anaerobic conditions and the final results were expressed as CFU/g.

In an embodiment, the process of obtaining spray-dried encapsulated A. muciniphila DSM 22959 suspended in skim milk matrix was conducted under aerobic conditions. This presents a great advantage considering its industrial application. As shown in Table 1 below, the viability of the A. muciniphila obtained using the method of the present disclosure, determined by colony-forming units count, at 4° C. was maintained 4 weeks under aerobic conditions. Moreover, aerobic storage at 23° C. for the same period showed promising results in terms of viability, which is an advantage in terms of transportation logistics, and shelf-life in household storage conditions.

As shown in Table 1 below, one of the advantages of the method of the present disclosure is that the spray-dried encapsulated A. muciniphila obtained can be stored under aerobic conditions while maintaining high viability levels. For example, Marcial-Coba et al (2018) demonstrated a reduction of at least a 6-log after 4 weeks of aerobic storage at 25° C. Comparatively, the spray-dried encapsulated A. muciniphila obtained using the method of the present disclosure shows only a 3-log reduction, maintaining a cell viability of ˜10⁶ CFU/g under the same storage conditions. In another study, the same authors incorporated their previously immobilized cells into a food matrix (Marcial-Coba et al 2019). However, the storage effect was only evaluated under anaerobic conditions which does not correspond to a feasible storage modality (more expensive, not suitable for a household context). Regarding the work of van der Ark and colleagues (2017), a sharp reduction in viability (3-log reduction) was found only after 3 days of refrigerated storage. Lastly, Chang et al (2020) used the spray-drying technique to encapsulate and immobilize A. muciniphila strain 139 cells in modified alginate microgels that were produced under anaerobic conditions. They reported a decreasing viability tendency during anaerobic storage for 12 days at 4° C. using a MTT assay for viability assessment. However, it should be recalled that the MTT assay measures bacterial metabolic activity without reflecting the true viable cell numbers available (biomass), a crucial factor for industrialisation (scaling-up).

TABLE 1
Viability of spray-dried A. muciniphila DSM 22959 in skim milk matrix
after 28 days of aerobic storage at 4° C. and 23° C. (present
proposal) as compared with results available in the literature.

| | | | | | | log |
|---|---|---|---|---|---|---|
| | | Storage | | | | reduction |
| | | Timepoint | Temperature | | | (to Day 0 |
| | Technology | (Days) | (° C.) | Atmosphere | Range CFU/g | value) |
| Present | Spray-drying | 0 | — | Aerobiosis | 3.23 × 10⁷- | — |
| proposal | | | | | 1.20 × 10⁸ | |
| | | 28 | 4 | Aerobiosis | 1.08 × 10⁷- | 0-1 |
| | | | | | 4.82 × 10⁷ | |
| | | | 23 | Aerobiosis | 4.25 × 10⁵- | 1-3 |
| | | | | | 1.17 × 10⁶ | |
| Marcial- | Extrusion + | 0 | — | n.a. | ~10⁹ | — |
| Coba et | Freeze drying | 30 | 4 | Aerobiosis | ~10⁷-10⁸ | 1-2 |
| al. (2018) | | | | Anaerobiosis | ~10⁸ | 1 |
| | | | 25 | Aerobiosis | <10³ (below | >6 |
| | | | | | detection limit) | |
| | | | | Anaerobiosis | ~10⁴-10⁵ | 4-5 |
| Marcial- | Extrusion + | 0 | — | n.a. | ~10⁷ | — |
| Coba et | Freeze drying + | 30 | 4 | Anaerobiosis | ~10⁷ | 0 |
| al. (2019) | Chocolate | | 15 | Anaerobiosis | ~10⁷ | 0 |
| Van der | Double | 0 | — | n.a | 2.5 × 10⁶- | — |
| Ark et al. | emulsion | | | | 1.2 × 10⁷ | |
| (2017) | (W/O/W) | 3 | 4 | Aerobiosis | ~99.9% log | 3 |
| | | | | Anaerobiosis | reduction, | 3 |
| | | | | | equivalent to a | |
| | | | | | 3-log reduction | |
| | | | | | (only normalized | |
| | | | | | data was provided) | |

The term “comprising” whenever used in this document is intended to indicate the presence of stated features, integers, steps, components, but not to preclude the presence or addition of one or more other features, integers, steps, components, or groups thereof.

The disclosure should not be seen in any way restricted to the embodiments described and a person with ordinary skill in the art will foresee many possibilities to modifications thereof.

The embodiments described above are combinable.

The following claims further set out particular embodiments of the disclosure.

1. An encapsulated anaerobic bacteria obtained by spray-drying and encapsulation in a solution of milk derived products; preferably a solution of milk-derived dry products, wherein the encapsulated anaerobic bacteria are selected from Akkermansia muciniphila; Anaerobutyricum hallii; Faecalibacterium prausnitzii; wherein Faecalibacterium prausnitzii is one of the following: Faecalibacterium duncaniae, Faecalibacterium hattorii, Faecalibacterium gallinarum, or mixtures thereof.

2. The encapsulated bacteria according to claim 1 wherein the bacterium is Akkermansia muciniphila DSM 22959.

3. The encapsulated anaerobic bacteria according to claim 1 wherein the solution of milk derived product is a skim milk.

4. A particle comprising a capsule, wherein said capsule comprises a solution of milk derived products, preferably milk-derived dry products, in the shell and an anaerobic bacterium in the core, wherein the bacterium is selected from: Akkermansia muciniphila, Anaerobutyricum hallii, Faecalibacterium prausnitzii; wherein Faecalibacterium prausnitzii is one of the following: Faecalibacterium duncaniae, Faecalibacterium hattorii, Faecalibacterium gallinarum, or mixtures thereof, wherein the amount of the solution of milk derived products ranges from 5-20% (v/v).

5. (canceled)

6. The particle according to claim 4 wherein the bacterium is Akkermansia muciniphila DSM 22959.

7. The particle according to claim 4 wherein the quantity of anaerobic bacteria ranges from 10⁵ to 10¹⁰ CFU/g.

8. The particle according to claim 4 wherein the solution of milk derived products is a skim milk.

9. A foodstuff composition comprising the particle and/or the encapsulated anaerobic bacteria described in claim 1 and a suitable food matrix and/or viable anaerobic bacteria in a chocolate matrix.

10. The foodstuff composition according to the previous claim 9 wherein the suitable food matrix is selected from: dairy products, cereal matrix, oil matrix, sugar matrix, chocolate matrix, soy milk matrix, juice matrix, vegetable matrix and fruit matrix.

11. The foodstuff composition according to claim 9 wherein the amount of particle and/or the encapsulated anaerobic bacteria ranges from 10⁶-10¹¹ CFU/serving, preferably 10⁷-10⁹ CFU/serving.

12. The foodstuff composition according to claim 9 wherein the bacteria are viable at least after 12 days of aerobic storage conditions at 4° C.-25° C.; preferably wherein the bacteria are viable at least after 4 weeks of aerobic storage conditions at 4° C.-25° C.

13. Foodstuff comprising the composition described in claim 9.

14. Foodstuff according to the previous claim 13 wherein the foodstuff is cheese, yogurt, ice-cream, plant-based yoghurt-type product, beverage, juice, chocolate food supplement, cereal bar.

15. A method for obtaining the encapsulated bacteria according to claim 1 comprising the steps of:
obtaining a sample of anaerobic bacteria;
adding a solution of milk-derived dry product to the sample of bacteria to obtain a bacteria suspension;
spray-drying the suspension to obtain the encapsulated bacteria.

16. The method according to the previous claim 15 wherein the anaerobic bacteria is selected from Akkermansia muciniphila, Anaerobutyricum hallii, Faecalibacterium prausnitzii, wherein Faecalibacterium prausnitzii is one of the following: Faecalibacterium duncaniae, Faecalibacterium hattorii, Faecalibacterium gallinarum, or mixtures thereof.

17. The method according to claim 15, wherein the bacteria is Akkermansia muciniphila DSM 22959.

18. The method according to claim 15 wherein the solution of milk-derived dry product is a skim milk.

19. The method according to claim 15, wherein the spray-drying conditions are: an inlet temperature from 140-180° C.; an outlet temperature from 55-80° C.; and a flow rate from 5-20 ml/min.

20. Encapsulated bacteria or particle according to claim 1, for use as a live biotherapeutic product or food additive.

21. Encapsulated bacteria or particle according to claim 1, for use as a probiotic.
